(19) United States
(12) Patent Application Publication
(10) Pub. No.: US 2006/0046123 A1
(43) Pub. Date: Mar. 2, 2006

(54) Passive fluid pump and its application to liquid-feed fuel cell system

(76) Inventors: Zhen Guo, Storrs, CT (US); Yiding Cao, Miami, FL (US)

Correspondence Address: YIDING CAO, 16302 SW 68 Terrace, Miami, FL, 33193, US

(21) Appl. No.: 10/924,942

(22) Filed: Aug. 24, 2004

(51) Int. Cl. H01M 8/02; A61L 9/04

(52) U.S. Cl. 429/34; 429/39; 239/44

(57) ABSTRACT

Methods and devices are disclosed for transferring a first liquid into a second liquid through a wick material. Said wick material preferentially has a higher wicking capability with respect to the first liquid than to the second liquid, and is disposed in a siphon fashion with the first or intake end contacting the first liquid and the second or discharge end contacting the second liquid. Because of the different wicking capabilities, a net amount of the first liquid is pumped into the second liquid. The device described above is used as a fuel delivery means for a liquid-feed fuel cell system, which directly utilizes a liquid fuel without an intermediate reforming process, such as a direct methanol fuel cell (DMFC). In this case, a methanol fuel and an aqueous methanol solution are stored separately in two containers and a wick is disposed between the two containers in a siphon fashion, with the container of the aqueous methanol solution communicating with the anode of the DMFC. Methanol is siphoned from the methanol container to the aqueous solution container in-situ when the methanol in the aqueous methanol solution is consumed during the operation of the fuel cell. Through a proper selection of the wick and the containers, the methanol concentration near the anode of the DMFC is maintained within a preferable range.

### TECHNICAL FIELD

This invention relates in general to a pumping device, and more particularly to a passive fluid pump, using the capillary pressure difference between different liquids in a wick to generate a fluid motion. The device can serve as a fuel delivery means for a fuel cell system, particularly, for a liquid-feed fuel cell system.

### BACKGROUND OF THE INVENTION

This invention relates to devices which can be used to dispense a fluid into another fluid at a small flow rate. Micro fluid pumps are commonly used for this purpose. Many micro-pumps of prior arts utilize electromechanical mechanisms to produce a driving pressure head. For example, micro-pumps utilizing piezoelectric materials are known wherein a pump element is oscillated by the application of electrical impulses on piezoelectric crystals to create a pressure differential in a liquid. U.S. Pat. Nos. 6,283,730 and 6,247,908 disclose such micro-pumps. However, piezoelectric micro-pumps are relatively complex and expensive to manufacture on a small scale necessary to control a small flow rate and require high maintenance costs during operations.

Furthermore, micro-scale fluid pumps mentioned above are all electricity-consuming devices. These micro-pumps are unsuitable for the applications in which electricity is precious and power-consuming components are to be avoided. For example, micro-pumps are considered to be used in lab-on-a-chip devices, devices for biological support purposes, devices which deliver fuel for direct methanol fuel cells, and other pumping applications in handheld systems. In these working environments, devices have to be miniaturized to a handheld size and they are always limited in how long they can operate as truly portable (i.e. unplugged) devices by the quantity of energy stored within them. One avenue leading to further miniaturization of these handheld systems and extending their operating time is to eliminate as many power-consuming and otherwise complex elements as possible, and to replace them with passive components that operate via such natural power sources as gravity, air pressure, absorption, capillary forces, or simple manual attention. Clearly, there is a need for a micro-pump which is capable of transporting a liquid at a small flow rate without any moving part and without requiring any external power source. Ideally, such a micro-pump should be simple in construction, and all of the components of the pump should be manufactured from relatively inexpensive, and easily workable materials.

One such passive pump is a siphon, which utilizes the siphoning action to transfer a liquid form a higher container to a lower container such as taught in U.S. Pat. Nos. 6,412,528 and 4,112,963. The prior arts also showed apparatuses which use porous media for moving, transferring, supplying, or dispensing liquids to lower levels by the siphoning action (see U.S. Pat. Nos. 4,759,857; 2,770,492; 5,006,264; 5,329,729; and 3,069,807). In these applications, a wick capable of wicking a liquid is positioned in a siphon fashion, which functions similarly to the suction tube used in siphons. Devices of this general class will hereafter be referred to as “capillary siphon”, although it should be kept in mind that the shape of the wick is not a matter of concern.

Although siphons and capillary siphons have similar configurations and all depend upon the hydraulic pressure gradient created by the difference in vertical levels between the intake end and discharge end to force a liquid to move, it should be emphasized that the mechanisms to cause the liquid to go upward as part of the siphoning process are different between the siphons and capillary siphons. For the siphon, it is the pressure of the atmosphere that forces the liquid to move upward along the suction pipe immersed in the higher container. By contrast, capillary siphons rely on capillary action to raise liquid from the higher container into the wick. Once the liquid reaches the top portion, a very slow process of capillary action, gravity will pull the liquid down toward the outlet end of the wick. Although normally one would prime the capillary siphon by saturating the wick, this is unnecessary, as the capillary siphon with a properly selected wick can self-prime. The wick mentioned above may be made of a synthetic or nature porous material as long as it can provide a sufficient capillary action naturally. Examples of the wick materials are papers, cloths, ceramic fibers, carbon fibers, and glass fibers.

The configuration of a capillary siphon mentioned above is shown in FIG. 1. The heart of the capillary siphon is a strip of wick 100 positioned in a siphon fashion, by immersing one end in a liquid 30 in container 40 and allowing the liquid to drain from the other end, which extends below the liquid level of container 40. A suitable container 50 is positioned to collect the siphoned liquid 30. Container 40 and trough 50 are sealed with plugs 10 and 10b, respectively. A sleeve tube 20 is used to prevent evaporation of the liquid when the wick hung open in the air. The wick 100, which contains multiple interconnected pores, raises the liquid 30 from the higher reservoir 40 into the porous media through the capillary action. Once the liquid reaches the top portion, gravity will pull the liquid down toward the outlet end of the wick 100 through the gravitational pressure head ρgh, where ρ is the density of the liquid, g is the gravitational acceleration, and h is the vertical distance of liquid levels between the container 40 and container 50.

While liquid transportation in a capillary siphon could occur in a wide variety of situations, which includes initial contact of a dry wick with liquid, liquid flow through a fully saturated wick, and removal of a liquid from a wick, the transport phenomenon can be described by a single process—liquid flow response to a capillary pressure and gravitational head. This process may be described mathematically by the Darcy's equation:

$$V = \frac{K}{μ} ( \frac{Δ {P}_{c} + ρ gh}{L} ) \qquad ( 1 )$$

where V (cm/s) is the apparent velocity of the liquid (volume flow rate divided by cross-sectional area), K (cm²) is the permeability that describes the ease with which liquid flows through wicks, μ (g/cm s) is the viscosity of the advancing liquid, ρ is the liquid density (g/cm³), g is the gravitational acceleration (cm/s²), L=ΔL+ΔH+h is the total liquid transfer length (cm), and ΔP_{c} (g/cm s²) is the capillary pressure. The magnitude of the capillary pressure is described by the Laplace equation as applied to an idealized capillary tube:

$$Δ {P}_{c} = \frac{2 γ cos θ}{{R}_{c}} \qquad ( 2 )$$

where γ is the surface tension of the advancing liquid (dyn/cm or mN/m), θ is the contact angle at the liquid/solid/air interface, and R_{c} is the radius of the tube (cm). Thus, wick with a smaller contact angle will have a larger capillary pressure or a larger pressure difference to drive the liquid movement. As the saturation increases from zero, the liquid will fill the smallest pores first. At a low saturation, capillary pressure can be very large because of a very small R_{c}. The capillary pressure decreases with an increase in saturation as the pores fill with liquid, and decrease to zero for a completely saturated wick. Permeability also varies greatly with saturation, being nearly zero at low saturation and increasing as the pores being filled with liquid.

The prior arts described above in connection with a capillary siphon are primarily for transporting a single fluid (the same substance) from a higher level to a lower level. They also lack a mechanism to easily and quickly control the flow rate of the liquid from one container to another when desired. Obviously, these capillary siphons are not intended to transport a fluid of given substances to a solution in which a preferable concentration range of the substance (or substances) delivered is maintained. Therefore, these capillary siphons cannot serve as a passive micro pump for the fuel delivery purpose of a portable power generation device, which often requires the liquid delivery system to work at an arbitrary orientation.

### SUMMARY OF THE INVENTION

It is therefore an object of the present invention to develop a pumping device which transports a first liquid (or the first solution) into a second liquid (or the second solution) through a wick material. Said wick material preferentially has a higher wicking capability with respect to the first liquid than for the second liquid (said wick material preferentially wicks the first liquid better than the second liquid), and is disposed in a siphon fashion with the first or intake end in contacting with the first liquid and the second or discharge end in contacting with the second liquid. Because of the different wicking capabilities, a net amount of the first liquid is pumped into the second liquid. The passive pump having the aforementioned function is referred to as the bi-liquid capillary siphon in the present invention.

Another object of the present invention is to provide a method of controlling the flow rate of a liquid through the wick when desired. The permeability of a wick generally depends on the external force that is applied to the wick, and can be adjusted through adjusting the compression force upon the wick. The control of the fluid flow through the wick is easily achieved through a flow control pinch valve that is mounted on the wick.

Yet another object of the present invention is to develop a fuel storage and delivery assembly for a fuel cell system which directly utilizes a liquid fuel without an intermediate reforming process, such as a direct methanol fuel cell (DMFC). In this case, a methanol fuel and an aqueous methanol solution are stored separately in two containers and a wick is disposed between the two containers in a siphon fashion, with the container of the aqueous methanol solution communicating with the anode of the fuel cell. Methanol is siphoned from the methanol container to the aqueous methanol solution container in-situ when the methanol in the solution is consumed during the operation of the fuel cell. Through a proper design of the wick and the containers, the methanol concentration near the anode of the fuel cell is maintained within a preferable range.

Yet another object of the present invention is to develop a compact liquid-feed fuel cell system which has a disposable fuel storage and delivery assembly. Said fuel storage and delivery assembly has an aqueous solution chamber and a fuel chamber which are coaxially positioned therewith and communicate with each other through at least one wick material. Upon insertion of the fuel storage and delivery assembly into the fuel cell system, the aqueous methanol solution chamber begins to communicate with the space adjacent to the anode of the fuel cell through a special opening mechanism. After the fuel in the fuel container is consumed, the fuel storage and delivery assembly can be easily removed from the fuel cell system and a new fuel storage and delivery assembly is installed.

### BRIEF DESCRIPTION OF THE DRAWINGS

Other features and advantages of the present invention will become apparent from the following description of the preferred embodiments of the invention, and the accompanying drawings, wherein:

FIG. 1 is a schematic sectional view of a conventional capillary siphon as practiced in the prior art.

FIG. 2 is a schematic sectional view of an embodiment of the invention wherein a wick, which is preferentially wetted better by the first liquid than the second liquid, is positioned in a siphon fashion with an intake end placed in the first liquid and a discharge end in the second liquid.

FIG. 3 is a plot of the liquid level differences between the first and the second containers versus time for a number of wicks made of different kinds of materials.

FIG. 4 is a schematic sectional view of another embodiment of the invention showing a close-looped wick configuration.

FIG. 5 is a schematic sectional view of another embodiment of the invention showing a flow control pinch valve mounted on the wick to control the flow rate through the wick.

FIG. 6 is a schematic sectional view of an embodiment of the present invention in which a fuel storage and delivery assembly is employed for a direct methanol fuel cell.

FIG. 7 is a schematic sectional view of another embodiment of the present invention in which a fuel storage and delivery assembly is employed for a direct methanol fuel cell and in which the fuel is directly delivered to the anode of a MEA.

FIG. 8 is a plot showing the output voltage curves of a fuel cell with the fuel storage and delivery assembly and the same fuel cell without the fuel storage and delivery assembly. The external load used in these experiments is a small fan.

FIG. 9 is a schematic sectional view of another embodiment of the present invention in which the fuel storage and delivery assembly is employed for a direct methanol fuel cell, and in which a close-looped wick is employed.

FIG. 10 is a schematic sectional view of another embodiment of the present invention in which the fuel storage and delivery assembly is employed for a direct methanol fuel cell, and in which a close-looped wick directly delivers the fuel to the anode of a MEA.

FIG. 11A is a schematic sectional view of a compact direct methanol fuel cell having a disposable fuel storage and delivery assembly.

FIG. 11B is a schematic cross-sectional view taken along the lines 11B-11B of FIG. 11A.

### DETAILED DESCRIPTION OF THE INVENTION

To understand the working mechanisms of a bi-liquid capillary siphon, the wicking phenomenon of a wick material with respect to different liquids is first discussed. It is well known that a liquid wet some solids and do not others. The contact angle, which is the angle between the edge of the liquid surface and solid surface, measured inside the liquid, is a measure of the quality of wetting. We normally say that a liquid wets a surface if contact angle is less than 90° and does not wet if contact angle is more than 90°. Values of contact angle less than 20° are considered strong wetting, and values of contact angle greater than 140° are strong nonwetting. Water on clear glass represents a wetting case. Water on Teflon or mercury on clean glass represents a nonwetting case. It is generally found that liquids with low surface tensions easily wet most solid surfaces resulting in a zero contact angle, which means that the molecular adhesion between solid and liquid is greater than the cohesion between the molecules of the liquid. Liquids with high surface tensions mostly give a finite contact angle, and here the cohesive forces become dominant.

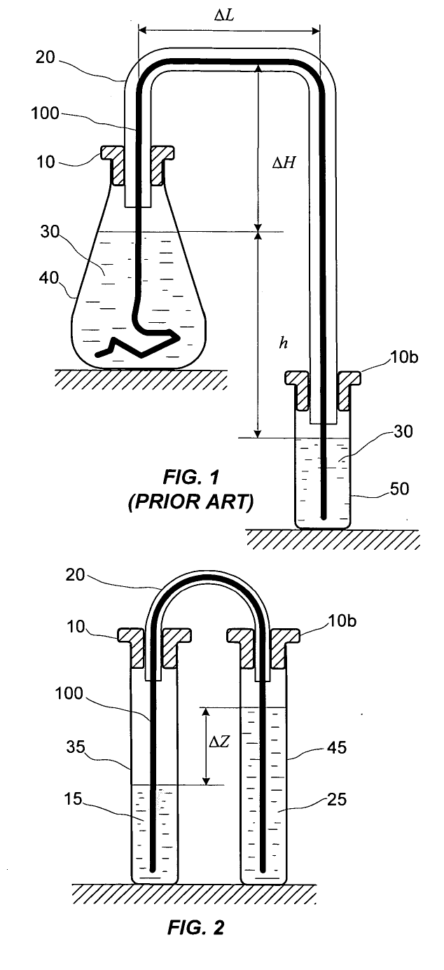

The surface tension of water is 72.75 mN/m at 20° C.; and common organic liquids have surface tensions around 20-30 mN/m. We can expect that organic liquids, such as methanol and ethanol, preferentially wet most solid surfaces better than water. Teflon is highly hydrophobic (not wetted by water), but it can be completely wetted by a low surface tension liquid (such as methanol and ethanol). If a strip of Teflon tape is used as the wick of the capillary siphon as shown in FIG. 1 and the testing liquid 30 is methanol, methanol will rise along the vertical surface of the Teflon. Once methanol reaches the top portion, a very slow process of the capillary action, gravity will pull the methanol down toward the outlet end of the wick. As a result, methanol was transferred from container 40 to container 50. If the testing liquid 30 is changed to water, the water will be depressed along the vertical surface of the Teflon. Consequently Teflon tape cannot wick water; it would not establish a good siphon for water.

As mentioned in the previous sections, one of the objectives of the present invention is to provide methods and devices for transporting a first liquid of given substance (or substances) into the second liquid of different substance (or substances) through a wick material. In order to achieve this goal, the principles of capillary siphons are utilized in a novel way as shown in FIG. 2. A wick material 100 is disposed in a siphon fashion with an intake end in contacting with the first liquid 15, which preferentially wets wick 100, and a discharge end in contacting with the second liquid 25, which wets the wick 100 weakly. The wick 100 comprises a porous material from a group of materials consisting of ceramic, fiberglass, carbon fiber, polymers, and cotton. The net mass transfer between containers 35 and 45 will thus be determined by two competing capillary-pressure driven flows in the wick 100. Because the capillary pressure of the liquid 15 is higher than that of liquid 25, it is expected that the apparent velocity of the liquid 15 is higher than that of liquid 25. Therefore, the liquid 15 will reach the top portion of the wick 100 first. Then the gravity force will accelerate the movement of liquid 15 and sweep the liquid 25 down into container 45. Apparently, the transportation process of the liquid 15 dominants the liquid movement in wick 100, and, as a result, liquid 15 is pumped from container 35 to container 45.

For a better understanding of the present invention, methanol and water were used as the testing liquids in an experiment. It should be noted that the scope of this invention is not limited to these two liquids. The experiment setup was similar to the bi-liquid capillary siphon shown in FIG. 2. Two glass tubes, with an outer diameter of 13 mm, an inner diameter of 10 mm, and a length of 100 mm, were arranged vertically side by side. One of the tubes was denoted as container 35 and the other one as container 45. Methanol and water were added to container 35 and container 45 respectively to the same vertical level. Four wicks made of different kinds of materials were used in the experiment. The experiment was done at a room temperature of 20° C. Within the first few hours of the experiment, the liquid level in container 35 decease, and the liquid level in container 45 increases. The vertical difference of the two liquid levels was denoted as ΔZ in FIG. 2. This indicated that methanol in container 35 was pumped into water in container 45. In the first few weeks, the vertical difference of the two liquid levels reached their maximum values, which was followed by a very slow process of decreasing. After two months, most vertical differences of the two liquid levels became nearly stable.

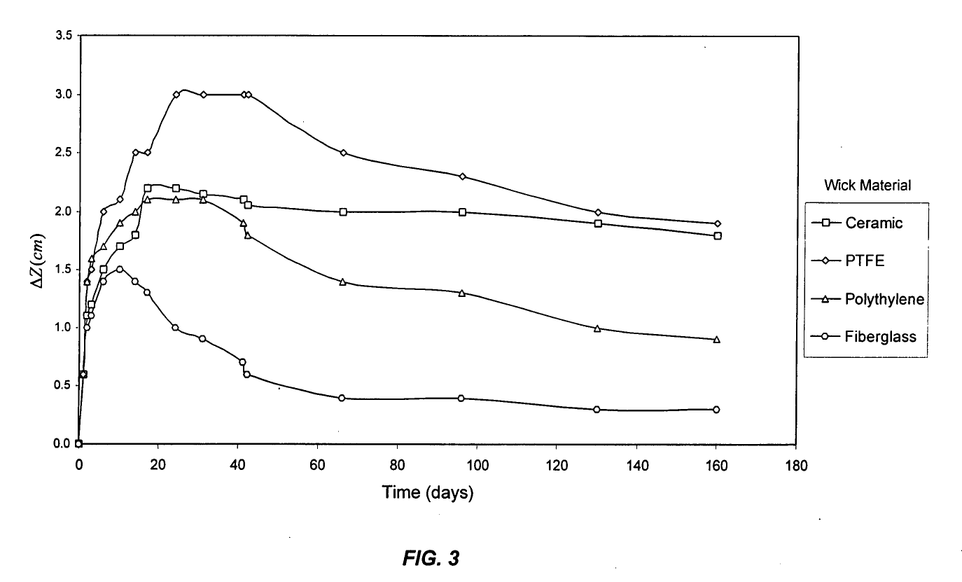

In FIG. 3, which is a plot of the vertical differences of the two liquid levels measured with a number of wicks versus time. The plateaus of vertical differences of the two liquid levels are of principal interest. The wicks used in the test are made of a wide variety of materials ranging from high-energy surface to low-energy surface media, which include fiberglass, Nextel® 440 ceramic fiber, polyethylene fiber and PTFE tape. Some specifications of these wicks are shown in Table 1. Fiberglass is hydrophilic (small water contact angle), whereas Nextel® 440 ceramic fiber, polyethylene fiber and PTFE tape are usually considered hydrophobic (large water contact angle). In theory, liquids having surface tensions lower than a solid critical surface tension should uniformly and completely wet the solid. Methanol with a low surface tension (γ=22.65 mN/m at 20° C.) is expected to wet most of these solids thoroughly (an exception is PTFE that has a critical surface tension of 18.5 mJ/m² at 20° C., which results in the contact angle between methanol and PTFE tape greater than 0°). On the contrary water with a higher surface tension (γ=72.75 mN/m at 20° C.) can only partially wet these solids.

TABLE 1
Some specifications of wicks

| Wick | Material | Dia. | Manufacturer |
|---|---|---|---|
| Fiberglass1286 | Fiberglass | ¼″ | Pepperell Braiding |
| | | | company |
| Nextel ® 440 | Ceramic* | 1/16″ | Omega Engineering |
| Sleeve | | | Inc. |
| Spectra ® Cable | Polyethylene | 0.060″ | Small Parts Inc. |
| PTFE Thread | PTFE | — | — |
| Seal Tape | | | |

*Ceramic fiber typical crystal type: gamma Al₂O₃ + mullite + amorph SiO₂

Equations (1) and (2) as discussed in the section of background of the invention can be used to explained the liquid movements in the bi-liquid capillary siphon as shown in FIG. 2. At the beginning of the experiment, the wick 100 was dry and ΔZ=0. Liquid 15 (methanol) preferentially wetted the wick 100 and capillary pressure of liquid 15 was larger than that of liquid 25 (water). At this moment liquid 15 penetrated through the wick 100 as it did in a single liquid capillary siphon (shown in FIG. 1). Because liquid 25 only weakly wetted or did not wet wick 100 at all, its penetration process was much slower than that of liquid 15. As a result, liquid 15 moved up quickly in wick 100. After it reached the top portion of wick 100, gravity would accelerate the penetration process of liquid 15 and it swept liquid 25 back into container 45. This resulted in a increasing in ΔZ and transportation of liquid 15 to liquid 25. After reaching its maximum value, ΔZ decreased slowly and liquid in container 45 (witch is a mixture of liquids 15 and 25 at this moment) was transferred slowly back to container 35. This phenomenon could be explained by the following two facts: adding liquid 15 to liquid 25 would normally increase the wettability of the latter with the wick 100, and as ΔZ increases gravity became a driving force to move liquid from container 45 to 35 in accordance with equation (1). When the combination of the static pressure, ρgΔZ, and the capillary pressure of the liquid in container 45 compensated the capillary pressure of liquid 15, ΔZ would reach its stable value. It is noted that the quasi-equilibrium value of ΔZ depended on the wick 100 and liquids 15 and 25. The cross-sectional areas of containers 35 and 45 have no influence on ΔZ. By inserting equation (2) into equation (1), the apparent velocities of liquids 15 and 25 in the wick 100 can be calculated. The quasi-equilibrium value of ΔZ can then be obtained by assuming that these two velocities are approximately equal at the quasi-equilibrium state.

$$Δ {Z}_{q - e} = \frac{2}{ρ {gR}_{wick}} ( {γ}_{1} cos {θ}_{1} - {γ}_{2} cos {θ}_{2} ) \qquad ( 3 )$$

where subscripts 1 and 2 refer to the first liquid (e.g., liquid 15) and the second liquid (e.g., liquid 25), respectively. To prevent the reverse flow mentioned above, a bi-liquid capillary siphon should work under such a condition that working value of ΔZ is lower than the quasi-equilibrium value of ΔZ. The bi-liquid capillary siphons could work in the following manner: when the liquid in container 45 (mixture of liquid 15 and 25) is consumed, the liquid level in container 45 decreases accordingly, which results in a decrease in ΔZ and subsequently a departure from the quasi-equilibrium state. This departure from the quasi-equilibrium state would induce a transfer of liquid 15 from container 35 to container 45. This process would continue until liquid 15 is exhausted.

From the results in FIG. 3, it is apparent that the ceramic wick used in the present test is advantageous in terms of practical applications. First, the ceramic wick exhibits a one-way transportation characteristic, i.e., it only allows methanol to flow from container 35 to container 45 and almost no water can flow back from container 45 to container 35. Second, the overshooting of the test curve is very small and its quasi-equilibrium value of ΔZ can be reached quickly. These features could allow the determination of the methanol concentration in the container 45 at the quasi-equilibrium condition if the bi-liquid capillary siphon is designed in a proper way as will be described later.

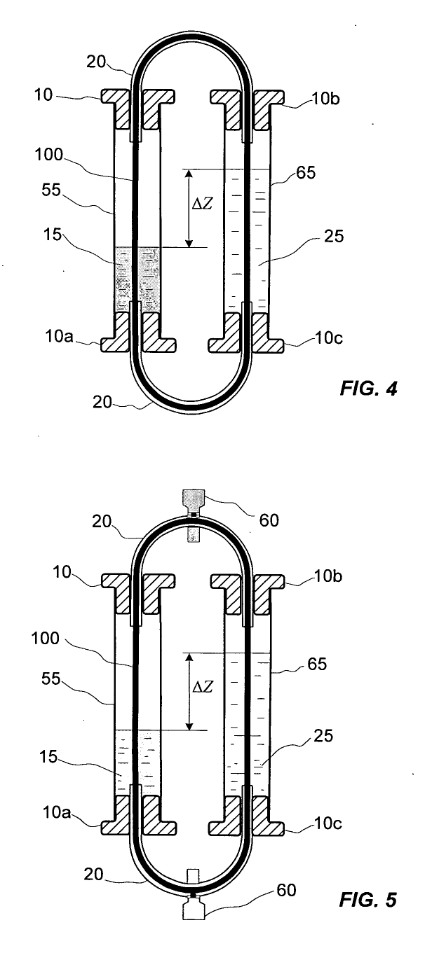

According to another embodiment of the present invention as shown in FIG. 4, container 55 has two open ends which are sealed with plugs 10 and 10a, respectively. Similarly, container 65 has two open ends which are sealed with plugs 10b and 10c, respectively. In this embodiment, wick 100 has a close-looped shape. Also the inner surface of sleeve tube 20 compresses the wick 100 tightly to prevent the liquid from flowing through the sleeve tube 20 when one of the sleeve tubes 20 is immersed in the liquid. The sleeve tubes 20 are selected from a group of materials of Nylon, Teflon and Polyethylene. The advantage of this embodiment is that the device can work at almost all orientations. FIG. 5 shows another embodiment of the present invention with an added control mechanism. Two flow control pinch valves 60 are mounted on the outer wall of the sleeve tubes 20. Through fine adjustments of the pinch valve 60, the compress force upon the wick 100 and consequently the permeability of the wick 100 can be adjusted. As a result, the flow rate through the wick 100 can be easily and reputably controlled.

The working mechanisms and embodiments of the bi-liquid capillary siphon have been described above. One of the most important applications of a bi-liquid capillary siphon is to the fuel storage and delivery assembly of a liquid-feed fuel cell such as a direct methanol fuel cell (DMFC). The direct methanol fuel cell has emerged as an attractive power source for portable devices because of its high energy density in generating electric power from fuel. Currently, one of the most fundamental limitations of direct methanol fuel cells is that the fuel supplied to the anode of the DMFC must be a very dilute aqueous methanol solution (usually 1˜2 M, which is translated into a methanol mass concentration of 3.2% to 6.4%). If the methanol concentration is too high, the methanol crossover problem would occur, which could significantly reduce the efficiency of the fuel cell and considerably shorten the life of the proton conductive membrane. If a DMFC is filled with a dilute aqueous methanol solution, the operation time of the fuel cell would be very short before a refueling is needed. This short operation time considerably diminished the advantage of a DMFC over a conventional battery. To overcome this difficulty, a complex fuel delivery system based on the modern microsystem technology was proposed. The proposed fuel delivery system would include micro-pumps, a methanol sensor, and a control unit such as that taught by U.S. Pat. Nos. 6,465,119 and 6,387,559. The fuel delivery system adds considerable costs to the fuel cell system and consume considerable amount of electricity from the fuel cell, which in turn significantly reduces the net power output of the fuel cell. As a result, the DMFC would have tremendous difficulty to compete with the conventional battery technology in terms of costs and power output. By incorporating the bi-liquid capillary siphon of the present invention to the DMFC, methanol and water can be carried separately and mixed in-situ during the fuel cell reaction, which provides a much simpler, cost effective, electricity free, and reliable fuel delivery system for direct methanol fuel cells.

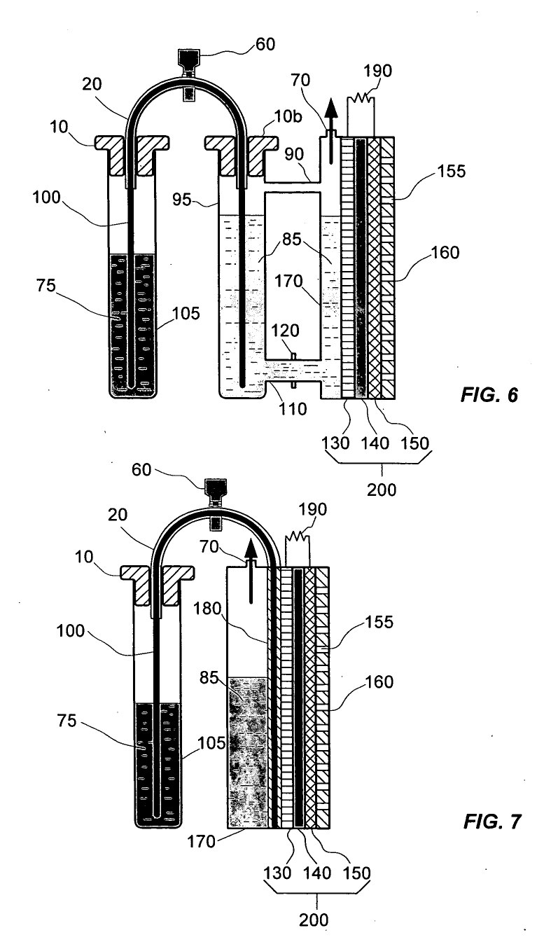

FIG. 6 shows a direct methanol fuel cell with which the fuel storage and delivery system of the present invention is used. The fuel storage and delivery system comprises a methanol container 105 and an aqueous methanol solution container 95, a wick 100 and a pinch valve 60 which is used to control the flow rate of the wick 100. Methanol 75 in container 105 is transported through the wick 100 to the container 95, where it is mixed with the water in the container 95 and forms an aqueous methanol solution 85. The container 95 may be connected to a fuel reservoir 170 through a connection 120. Therefore, an aqueous methanol solution is supplied to the anode 130 of the membrane electrode assembly (MEA) 200 which includes an anode 130, a membrane electrolyte 140 and a cathode 150. As the mixture of methanol and water is introduced into the anode 130 while oxygen (air) is introduced into the cathode 150 from the holes 155 in a fixture plate 160, reactions occur at anode 130 and cathode 150. As a result electrons flow from the anode 130 through the external load 190 to the cathode 150, while hydrogen ions flow from the anode 130 through the membrane electrolyte 140 to the cathode 150. As long as the chemical reactions continue, a current is maintained through the external load 190. Because of the chemical reactions, carbon dioxide will accumulate in the reservoir 170 as a reaction product. A CO₂ release mechanism 70 can be used to release carbon dioxide. The CO₂ release mechanism 70 could be in terms of a release valve. As the pressure is built up to a certain level, the release valve 70 is opened momentarily, which releases the carbon dioxide to the atmosphere. A short tube 90 can be used to connected the container 95 and the reservoir 170 to maintain a pressure balance between the container 95 and the reservoir 170. To operate direct methanol fuel cells at optimal conditions, the concentration of methanol in the reservoir 170 should be kept in a certain rang (for example, 1.0˜2.0 M). In theory, the consuming ratio between the methanol and water at an anode is 1:1. Departures from this value in practice occur frequently for many fuel cells. The value of this ratio for a particular fuel cell can be obtained from experimental studies. To maintain a preferred methanol concentration near the anode, the ratio of the methanol and water supplied to the anode of a fuel cell should be equal to the consuming ratio between the methanol and water at the anode. If the ratio of the cross-sectional areas of the methanol container 105 and the water container 95 are chosen to be equal to the consumption ratio of the methanol and water by the fuel cell, the consumed methanol could be complemented with the same amount. As a result, an approximately constant methanol concentration can be maintained at the anode.

FIG. 7 illustrates schematically a fuel cell system similar to that shown in FIG. 6 with an enhanced fuel delivery mechanism, in which a liquid permeating layer 180 is positioned proximately to the anode 130 and the wick 100 is attached to the liquid permeating layer 180. In this case, no separate aqueous methanol solution container is needed; the fuel reservoir 170 also serves as a solution container. The liquid permeating layer 180 is made of a material selected from a group of materials consisting of screen materials, non-woven fabrics, and woven fabrics as long as it has a capability of wicking a carbonaceous fuel/water mixture and has a large portion of pores to allow the carbon dioxide to vent out of the surface of anode 130. Inside the liquid permeating layer 180, dilute methanol aqueous solution moves upward due to the capillary action and methanol 75 moves downward through the wick 100. The diffusion of the methanol from the wick 100 to the liquid permeating layer 180 will keep the methanol concentration in the liquid permeating layer constant.

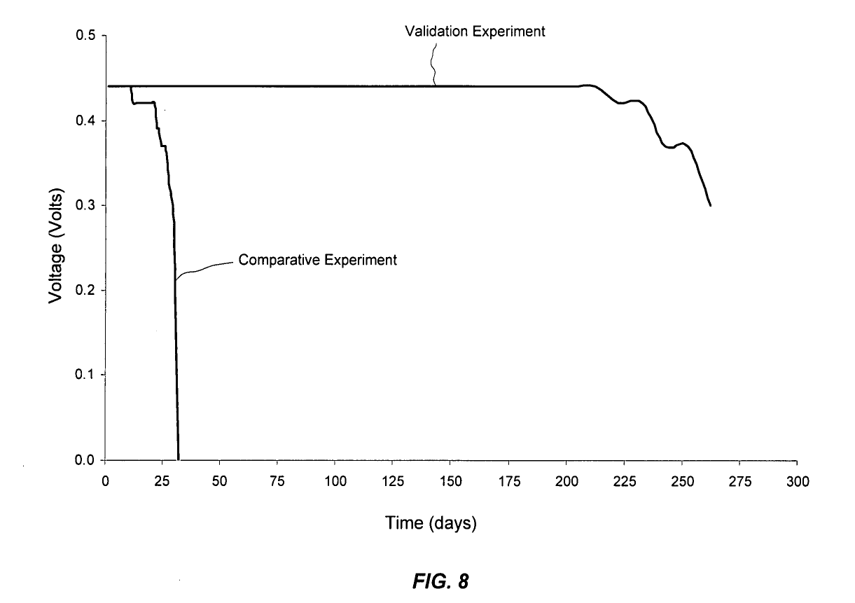

To validate the fuel storage and delivery system according to the present invention, a direct methanol fuel cell having a fuel storage and delivery assembly similar to that shown in FIG. 6 was used in a validation experiment. A fuel loading of 12 mL pure methanol was placed in the fuel container 105 and 20 mL of de-ionized water was filled in the water container 95. A small fan was used as the external load in the experiment. The output voltage of the fuel cell was shown in FIG. 8. The data reported here were obtained at a room temperature of 20° C. With a prolonged operation, the cell temperature became stable at 22° C. The fuel cell operated more than 260 hours until the methanol in the fuel container 105 was completely consumed. A comparative experiment was done without using the present fuel storage and delivery assembly. The same fuel cell was used to power the same small fan. A fuel load of 20 mL of 1.5 M methanol was directly placed in the fuel reservoir 170, and the fuel cell output voltage was recorded until the fuel cell output voltage was too low to power the fan. It is apparent from FIG. 8 that the fuel cell according to the present invention produced a stable output voltage for 11 days, whereas the same fuel cell in comparative experiment only produced an output voltage for less than 24 hours. The reason for the poor performance of the comparative experiment is that the concentration of the aqueous methanol solution in the fuel reservoir 170 continued to decrease as the methanol was consumed at the anode. In the validation experiment, however, methanol can be transferred continuously from the fuel tank 105 to the reservoir 170 through the wick 100 according to the methanol consumption rate of the fuel cell. As a result, the methanol concentration in reservoir 170 could be maintained within a preferred rang for a prolonged period of time until the methanol 75 in fuel container 105 is completely utilized by the fuel cell.

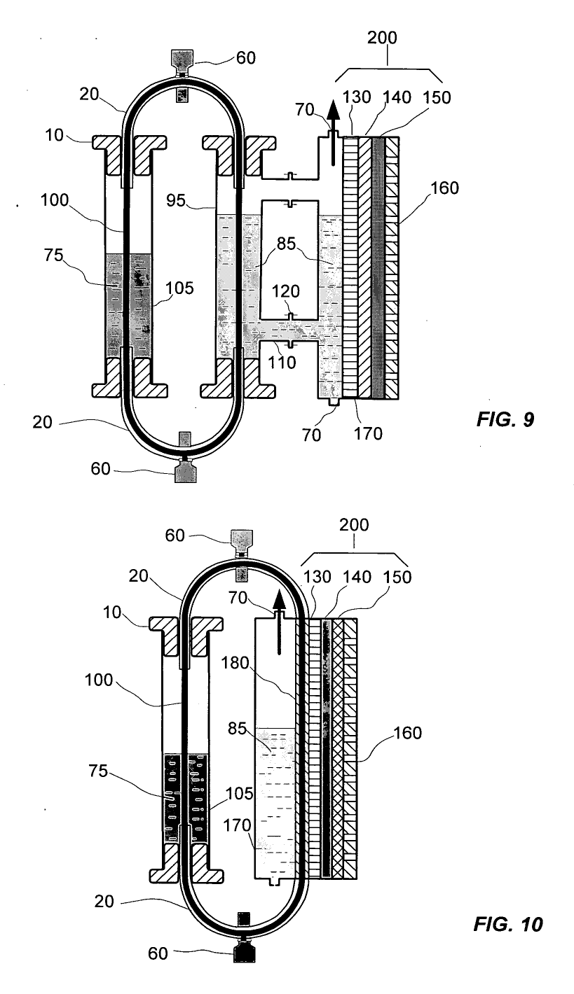

FIG. 9 illustrates an alternative embodiment of the present invention similar to the structure described in FIG. 6. In this embodiment, the wick 100 has a close-looped shape with the inner surface of the sleeve tubes 20 compressing the wick 100 tightly to prevent the liquid leakage through the sleeve tubes 20 when one of the sleeve tubes is immersed in the liquid. This embodiment can work at almost all orientations. FIG. 10 shows an alternative embodiment of the invention as described in FIG. 7 with a close-looped wick 100. Similar to the embodiment in FIG. 7, the liquid permeating layer will keep the anode side of the fuel cell adequately wetted.

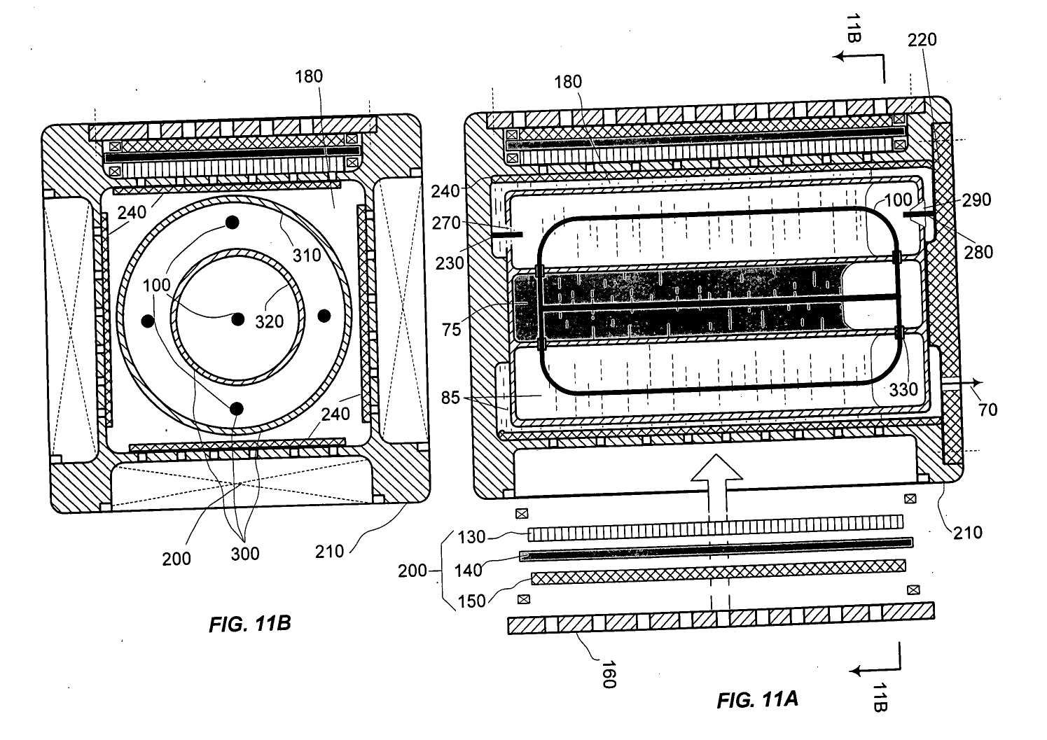

FIGS. 11A and 11B show a compact direct methanol fuel cell having a disposable fuel storage and delivery assembly 300 (FIG. 11B), which has an aqueous methanol solution chamber 310 and a methanol chamber 320 which are co-axially positioned therewith and communicate with each other through at least one wick 100. Sleeving tubes 330 are selected such that they will prevent the free mixing of methanol 75 and water methanol solution 85 (FIG. 11A) when the sleeve tubes are immersed in the liquid. Upon the insertion of the fuel storage and delivery assembly 300 into the fuel cell system and closing the top cover 220, an opening mechanism 230 creates an opening 270 on the bottom wall of the chamber 310. Then the chamber 310 begins to communicate with the reservoir 180 between the anodes 130 of MEAs 200 and the outer surface of chamber 310, so that the aqueous methanol solution 85 could flow into the reservoir 180 from the chamber 310. An opening 290 on the top wall of the chamber 310 is created through an opening mechanism 280 on the top cover 220 to equalize the pressure between the fuel storage and delivery assembly 300 and the reservoir 180. After the fuel in the chamber 320 in consumed, the fuel storage and delivery assembly 300 is removed from the fuel cell and a new fuel storage and delivery assembly is easily installed. In the case shown in FIGS. 11A and 11B, four MEAs 200 are disposed to the fixture 210 surrounding the fuel storage and delivery assembly 300. The anodes of the four MEAs 200 are arranged to face the fuel storage and delivery assembly 300 for the fuel supply purpose. Associated with each MEA, there is a compress plate 160 on the cathode side for assembling the MEA to the fixture and a liquid permeable layer 240 on the anode side in contact with the aqueous methanol solution 85 for the fuel spreading purpose. It should be noted that in the embodiment shown in FIGS. 11A and 11B, the fuel storage and delivery assembly 300 has a circular shape and the fixture 210 has a rectangular geometry. However, other shapes and geometries could be possible for a different design purpose. Also, the number of MEAs could vary for a different design.

Since many changes can be made in the construction of a capillary siphon to dispense a liquid at a small flow rate into a different liquid (some of which are mentioned above) and many apparent widely different embodiments of this invention could be made without departing from the scope thereof, it is intended that all mater contained in the above description or shown in the accompanying drawings shall be interpreted as illustrative and not in a limiting sense.

What is claimed is:

1. A device for transferring a first liquid into a second liquid of different substance (or substances) at a small flow rate comprising: a first vessel containing the first liquid and a second vessel containing the second liquid, and at least a wick preferentially being wetted by the first liquid and being positioned in a siphon fashion with the first portion contacting the first liquid and the second portion contacting the second liquid, the penetration rate of said first liquid in said wick is faster than the penetration rate of said second liquid in said wick, thereby a net amount of the first liquid is transferred into the second liquid.

2. A devices described in claim 1, wherein said wick comprises a porous material from a group of materials of ceramic, fiberglass, carbon fiber, polymers, and cotton.

3. A device described in claim 1 further comprises at least a sleeve tube mounted outside of said wick.

4. A device described in claims 1 and 3, wherein said sleeve tube is selected from a group of materials of Nylon, Teflon and Polyethylene.

5. A device described in claims 1 and 3 further comprises at least a flow control pinch valve mounted outside of the said wick, thereby the flow rate through said wick can be controlled through adjusting the pinch valve.

6. A device as described in claim 1, wherein said wick having a close-looped shape, thereby said device could work in many orientations.

7. A liquid-feed fuel cell system comprising:
at least a membrane electrode assembly (MEA), said MEA consisting of an anode, a membrane electrolyte, and a cathode; and an fuel storage and delivery assembly, said fuel delivery assembly comprising:
a fuel container filled with a carbonaceous fuel, a fuel reservoir filled with an aqueous solution of the carbonaceous fuel and communicating with said MEA for supplying a fuel-bearing fluid to said MEA, at least a wick preferentially wetted by the carbonaceous fuel and being positioned in a siphon fashion with the first portion contacting the carbonaceous fuel and the second portion contacting the aqueous solution of the carbonaceous fuel, the penetration rate of said carbonaceous fuel in said wick is faster than the penetration rate of said aqueous solution of the carbonaceous fuel in said wick, thereby, the carbonaceous fuel is transferred into the aqueous solution of the carbonaceous fuel in-situ when the carbonaceous fuel in said fuel reservoir is consumed by the reactions at the MEA of said fuel cell system.

8. A fuel storage and delivery assembly as claimed in claim 7, wherein said wick comprises a porous material from a group of materials consisting of ceramic, fiberglass, carbon fiber, polymers, and cotton.

9. A fuel storage and delivery assembly as claimed in claim 7 further comprises at least a sleeve tube mounted outside of said wick.

10. A fuel storage and delivery assembly as described in claims 7 and 9, wherein said sleeve tube is selected from a group of materials of Nylon, Teflon and Polyethylene.

11. A fuel storage and delivery assembly as claimed in claim 7 and 9 further compromises at least a flow control pinch valve mounted outside of the sleeve tube, thereby the flow rate through said wick can be controlled through adjusting the pinch valve.

12. A fuel storage and delivery assembly as claimed in claim 7, wherein said wick has a close-looped shape, thereby said fuel storage and delivery assembly can work in many orientations.

13. A fuel storage and delivery assembly as claimed in claim 7 further includes a liquid permeating layer configured to supply said aqueous solution of the carbonaceous fuel to the anode surface of said MEA, said liquid permeating layer being positioned proximately to the anode surface of the MEA and in the fuel reservoir.

14. A fuel storage and delivery assembly as claimed in claim 7 and 13, wherein said liquid permeating layer is made of a material selected from a group of materials consisting of screen materials, non-woven fabrics, and woven fabrics, which has capability of wicking carbonaceous fuel/water mixture and has a sufficiently large portion of pores to allow the carbon dioxide to vent out of the surface of the anode.

15. A fuel storage and delivery assembly as claimed in claims 7 and 13, wherein said wick is configured to supply the carbonaceous fuel to the liquid permeating layer, said wick being positioned proximately to or inside of the liquid permeating layer.

16. A compact liquid-feed fuel cell system comprising:
a fuel storage and delivery assembly, said fuel storage and delivery assembly comprising an inner chamber being filled with a carbonaceous fuel, an outer chamber being filled with an aqueous solution of the carbonaceous fuel and being co-axially disposed with said inner chamber, at least a wick preferentially being wetted by the carbonaceous fuel and being positioned in a siphon fashion with the first portion contacting the carbonaceous fuel and the second portion contacting the aqueous solution of the carbonaceous fuel;
at least a membrane electrode assembly (MEA) consisting of an anode, a membrane electrolyte, and a cathode, said anode facing said outer chamber; a fixture surrounding said outer chamber upon which said MEA or MEAs are disposed; a fuel reservoir between said outer chamber and said anode (or anodes); and an opening mechanism which could create an opening on the wall of said outer chamber, thereby, upon the installation of said fuel storage and delivery assembly, an opening on the wall of said outer chamber is created, and the aqueous solution of the carbonaceous fuel in said outer chamber flows into said reservoir between said outer chamber and said anode (or anodes), and thereby the carbonaceous fuel is transferred into the aqueous solution of the carbonaceous fuel in-situ when the carbonaceous fuel in said fuel reservoir is consumed by the MEA (or MEAs) of said fuel cell system.

17. A fuel storage and delivery assembly as described in claim 16, wherein said wick comprises a porous material from a group of materials consisting of ceramic, fiberglass, carbon fiber, polymers, and cotton.

18. A fuel storage and delivery assembly as described in claim 16 further comprises at least a sleeve tube mounted outside of said wick.

19. A fuel storage and delivery assembly as described in claims 16 and 18, wherein said sleeve tube is selected from a group of materials of Nylon, Teflon and Polyethylene.

20. A fuel storage and delivery assembly as described in claim 16, wherein said wick has a close-looped shape, thereby said fuel cell can work in many orientations.

21. A liquid-feed fuel cell as described in claim 7, wherein said carbonaceous fuel is methanol.

22. A liquid-feed fuel cell as described in 7, wherein said carbonaceous fuel is ethanol.

23. A compact liquid-feed fuel cell as described in claim 16, wherein said carbonaceous fuel is methanol.

24. A compact liquid-feed fuel cell as described in 16, wherein said carbonaceous fuel is ethanol.
